(19) United States
(12) Patent Application Publication
(10) Pub. No.: US 2014/0200536 A1
(43) Pub. Date: Jul. 17, 2014

(54) Leak proof equipment and method for the collection of stoma body wastes

(71) Applicant: Achilles Nassopoulos, Montreal, CA (US)

(72) Inventor: Achilles Nassopoulos, Montreal, CA (US)

(21) Appl. No.: 13/739,158

(22) Filed: Jan. 11, 2013

(51) Int. Cl. A61F 5/448

(52) U.S. Cl. CPC A61F 5/448
USPC 604/341; 604/338

(57) ABSTRACT

An apparatus and system for reducing the area of exposure of intestinal effluent on a stoma wafer by 90% to 100%. The apparatus consists of substituting the pouch on a ring of a mechanically connecting ring ostomy pouch and wafer assembly by a membrane. An orifice equal to or smaller than a stoma is made in the center of the installed membrane. An insert is passed with open end first through the top end of the membrane orifice, is pulled through the bottom end of the orifice, is overlapped by ¼ to ½ inches on the outer circumference of the mechanically connecting ring of the membrane. The membrane with the insert in position is slid down the stoma via the orifice, and the mechanically connecting ring of the membrane is mechanically connected to the opposite corresponding mechanically connecting ring of a stoma wafer that is adhering to the skin.

### CROSS-REFERENCE TO RELATED APPLICATIONS

| Document Number | | | |
|---|---|---|---|
| Country Code- | Date | | |
| Number-Kind Code | MM-YYYY | Name | Classification |
| A] US-4,681,574 A | 07-1987 | Eastman, Dianne | 604/344 |
| B] US-4,890,608 A | 01-1990 | Steer, Peter L. | 602/57 |
| C] US-2002/ | 03-2002 | Iseke, Jason W. | 604/338 |
| 0032418 A1 | | | |
| D] US-2003/ | 01-2003 | Bedard et al. | 604/332 |
| 0023210 A1 | | | |
| E] US-7,470,263 | 12-2008 | Strobech Esben | 604/336 |
| B2 | | | |
| F] US-2011/ | 07-2011 | Eakin, Paul Andrew | 604/339 |
| 0166539 A1 | | | |
| G] US-2012/ | 03-2012 | Laudick, David A. | 604/332 |
| 0078208 A1 | | | |
| H] US-8,366,695 | 02-2013 | Foley et al. | 604/342 |
| B2 | | | |

### BACKGROUND OF THE INVENTION

Colostomy is the surgical procedure of making an opening from the colon to the outside of the abdomen. In a colostomy, the surgeon may remove a segment of the colon, resulting in the colon being split into two separate parts. One end of the colon is passed through a small hole in the abdominal wall. This section of intestine called a stoma, allows for body waste to leave the body from the colon. The other end of the colon, which is attached to the rectum, may either be removed or closed off with sutures and left in the abdomen. There are other similar stoma operations such as ileostomy which is a stoma from the small intestine, and urostomy which is a stoma from the urinary system. Operations are performed for temporary or permanent stomas.

Such operations are performed on humans and animals as well. In all cases, stoma wafers are used; these are adhesive skin barriers or skin flanges with an opening at the center to allow the stoma to protrude. There are wafers that require cutting the center opening to custom fit the size of the stoma, there are wafers with a small opening of stretchable material at the center to custom fit to the stoma, and there are many other types of wafers for varied applications and conditions of stomas. One of the main goals of most stoma wafers is to prevent the contact of body waste with the skin. These stoma wafers consist of either a one piece wafer and bag or a two piece wafer with a snap-on or similar attachment for the bags. The bags snap on and serve to hold the body waste. Many other products related to stomas in preventing body waste leaks reaching the skin, or in protecting the skin from stoma leaks, or in cleaning the adhesive from the skin or in treating skin irritations due to the contact of body waste with the skin are in use and many more are being produced specifically for stomas. The conventional methods of stoma flanges or stoma wafers or stoma skin barriers and stoma pouches allow the body wastes to leave the stoma and to fall directly into the collecting pouch. Examples of conventional colostomy or other types of wafers are shown as examples of stoma wafers patented in U.S. Pat. No. 4,642,107, U.S. Pat. No. 4,681,574, U.S. Pat. No. 5,051,259, U.S. Pat. No. 6,206,864B1, and U.S. Pat. No. 6,764,473A When this process occurs there is direct contact of the body wastes with the stoma, since the stoma is totally exposed. The inner surface of the stoma wafer or skin barrier also comes into contact with the body wastes, especially if the body wastes are in liquid or semi-solid form. It is for that reason that the stoma wafers or stoma skin barriers concentrate mainly on preventing the body wastes that they are exposed to from going onto the skin.

In the absence of a barrier between the stoma wafer and the stoma pouch, or of a method which utilizes a disposable pouch whose inlet is fastened snugly around the stoma thus acting as a seal which prohibits the body waste from seeping out, it is inevitable that small leaks around the exposed stoma wafer will result in body wastes coming into contact with the exposed stoma, as well as with the skin surrounding it. The present invention has developed this specific equipment along with its particular method of use in order to prevent body wastes from the stoma of a patient from coming into contact with the skin, or from irritating the stoma itself.

The first part of three parts of the present invention serves to describe the equipment and the method of using a membrane (diaphragm) with a stoma size diameter center cut-out that is installed between a stoma wafer and a detachable body waste pouch in order to create a tight seal around the stoma thus preventing the contact of body waste with the skin. The membrane (stoma diaphragm barrier) of this invention could be fastened to the stoma body waste collecting pouch or to the stoma wafer, or it could be a loose membrane that is snapped or clamped in between the rings of the stoma body waste collecting pouch or bag and the stoma wafer. The membrane (stoma diaphragm barrier) of this invention that is to be used for one piece stoma wafer and body waste collecting pouch assemblies must be installed at the time of fabrication because the membrane (stoma diaphragm barrier membrane) has to be fixed between the stoma body waste collecting pouch and the stoma wafer adhering to the skin around the stoma of a patient.

The second part of three parts of the present invention elaborates on the first part of three parts of this invention in that an insert (disposable recyclable body waste collecting bag liner) such as the disposable stoma bag liner described in U.S. Pat. No. 5,423,782 is inserted, with closed end first, through the stoma size diaphragm barrier orifice, is then pushed through the center stoma diaphragm orifice into the interior of the stoma body waste collecting pouch, leaving a sufficient amount of the open end of the insert (disposable recyclable body waste collecting bag liner) out of the rim of the ring of the body waste collecting pouch in order to surround and to overlap over the exterior rim of the membrane (stoma diaphragm barrier membrane), also to overlap the exterior circumference of the mechanically connecting snap-on ring of the stoma body waste collecting pouch, and while they are being held in place, to mechanically connect by snapping together the membrane (stoma diaphragm barrier membrane) and the insert (disposable recyclable bag liner) inserted between the stoma body waste collecting pouch to the stoma wafer adhering around the stoma of a patient, thus creating a firm hold on the membrane (diaphragm barrier membrane) with a tight grip on the insert (disposable bag) around the stoma preventing body wastes from touching the skin. The third and final part of three parts of the present invention elaborates on the second part of three parts of the present invention in that the actual stoma body waste collecting pouch or bag of the mechanically connecting ring snap-on body waste collecting pouch and wafer system is cut out of the snap-on ring and the snap-on ring is refitted with the membrane (stoma diaphragm barrier) prefabricated as such in order to pass the insert (disposable recyclable body waste collecting bag) with open end first through the center stoma size orifice of the membrane (stoma diaphragm barrier), to pull the insert (recyclable body waste collecting bag) sufficiently through the stoma center orifice to overlap over the snap-on membrane (stoma diaphragm barrier) and to mechanically connect by snapping onto the stoma wafer adhering around the stoma of a patient, with the insert (disposable recyclable body waste collecting bag) snapped in snuggly around the stoma of a patient, thus preventing the contact of body excrements with the surface of the wafer and the skin.

Body wastes come in contact only at the extremity of the stoma that is exposed because the stoma protrudes partly out of the insert (disposable recyclable body waste collecting bag) that is held snuggly in place by the membrane (stoma diaphragm barrier). The body wastes and the malodorous air are prevented from escaping under the membrane (stoma diaphragm barrier).

### BRIEF SUMMARY OF THE INVENTION

It is an object of the present invention to create equipment and its corresponding methods of collecting body wastes or intestinal effluent from the stoma of a colostomy, ileostomy, or urostomy patient by which methods the body wastes do not come in contact with the skin of the patient by using a membrane (stoma diaphragm membrane) which is a membrane with a stoma size orifice at the center and which membrane overlaps the outer circumference of stoma wafers in three methods; the first method is by placing a membrane (stoma diaphragm barrier) between a conventional stoma wafer and a stoma pouch; the second method is by passing an insert (toilet disposable recyclable body waste collecting bag liner) with open end first through the stoma size orifice at the center of the membrane (stoma diaphragm barrier membrane) and pulling sufficient insert (recyclable liner) through the stoma size orifice so as to surround the outer circumference of the stoma membrane (diaphragm barrier membrane). The membrane (stoma diaphragm barrier membrane) must overlap the exterior rim of the ring of the stoma wafer, and the insert (recyclable liner) along with the membrane (stoma diaphragm barrier membrane) in place, are mechanically connected by snapping together between a conventional stoma wafer and a stoma pouch or bag. The third method is by replacing the conventional stoma body waste collecting pouch on the mechanically connecting snap-on ring of a stoma wafer and pouch snap-on assembly with the membrane (stoma diaphragm barrier) and passing an insert (recyclable liner) through the membrane (stoma diaphragm barrier) that is permanently fixed onto the mechanically connecting snap-on ring and by pulling a sufficient amount of the insert (recyclable liner) through the center stoma cut-out orifice of the membrane (stoma diaphragm barrier) in order to overlap over the outer diameter of the membrane (stoma diaphragm barrier) mechanically connecting snap-on ring which in turn is snapped onto the opposite corresponding mechanically connecting snap-on ring of the stoma wafer adhering around the stoma of a patient. Therefore I offer the equipment and its methods of use that:
1. Will be more sanitary and more hygienic than the existing conventional stoma wafer and pouch systems because the body wastes will not come in contact with the skin around the stoma of a patient.
2. Will be more environmentally friendly than the existing conventional stoma wafer and pouch systems because the use of stoma pastes, stoma seals, and other related equipment will no longer be required to prevent the body wastes from coming into contact with the skin around the stoma of a patient.
3. Will eliminate the use of conventional stoma body waste collecting un-disposable un-recyclable pouches by using only recyclable pouches that are disposable in the toilet.
4. Will eliminate skin irritations resulting from the contact of body wastes with the skin because there will not be any contact of body wastes with the skin around the stoma of a patient.
5. Will eliminate the use of glue pastes, glued stoma barrier rings, powders, creams and other ointments, to prevent and to treat skin irritations due to the contact of body wastes because there will not be any contact of body wastes with the skin around the stoma of a patient.
6. Will prolong the use of stoma wafers or stoma skin barriers because the surface usually vulnerable to body waste leaks on the stoma wafer will not come in contact with the body wastes of a stoma of a patient.
7. Will eliminate the use of pouch belts that are strapped around the waste of a patient with a stoma and are fastened to handles on the rim of the stoma body waste pouches or bags to keep body waste pouches that are heavily loaded with body wastes from detaching from the stoma wafer because the seal created between the snapped-on stoma diaphragm barrier of this invention and the stoma wafer adhering to the skin around the stoma of a patient holds the disposable recyclable stoma body waste bag solidly enough into place to make it very difficult to detach.
8. Will eliminate the need for a pouch belt that is strapped around the waist of a patient with a stoma and is fastened to handles on the rim of the stoma body waste pouch or bag in order to keep a body waste pouch that is heavily loaded with body wastes from detaching itself from the stoma wafer, because the seal created between the snapped-on stoma diaphragm barrier of this invention and the stoma wafer adhering to the skin around the stoma of a patient, holds the disposable recyclable stoma body waste bag solidly enough in place so as to make it very difficult for it to become accidentally detached.
9. Will allow the changing of disposable recyclable body waste bags in locations other than the toilet, such as the car, or on a chair, without any spillage of body wastes whether solid or liquid, and without the need of water or other cleaning agent.
10. Will considerably reduce the time needed for changing the disposable body waste bag or pouch or for cleaning the body wastes collected in body waste collecting pouches of stomas of patients.
11. Will enable the wearing of present conventional stoma wafers which leak or have sections that may not be adhering completely to the skin around a patient's stoma, while still preventing body wastes from leaking out of the stoma, as the present conventional stoma wafers will only be used for the purpose of anchoring the stoma barrier diaphragm and disposable recyclable body waste collecting bag liners and not for the purpose of sealing the area around the stoma.

### BRIEF DESCRIPTION OF THE SEVERAL VIEWS OF THE DRAWING

FIG. 1 is the front and back of a two-piece snap-on type ring mechanically connecting colostomy pouch with Velcro opening.

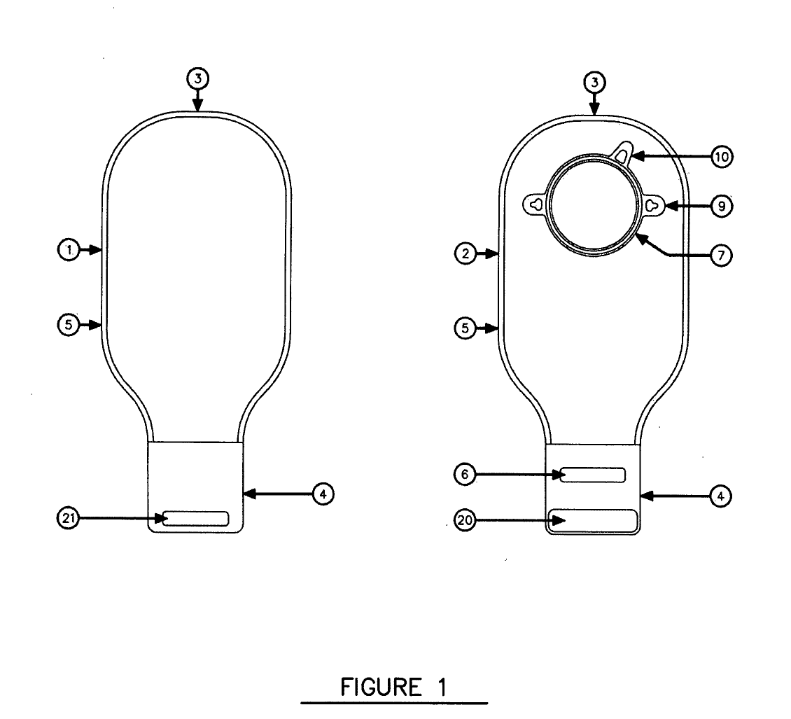

FIG. 2 is the front and back of a two-piece snap-on type ring mechanically connecting colostomy adhesive wafer or flange.

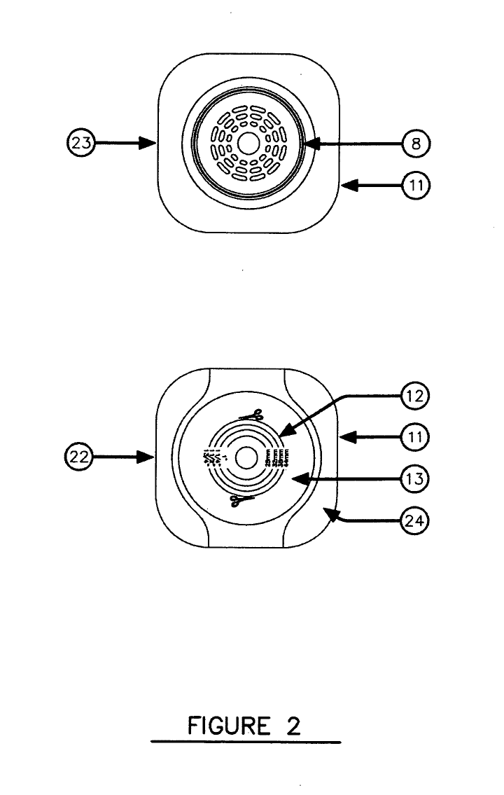

FIG. 3 is the front and back of the stoma membrane (diaphragm barrier membrane).

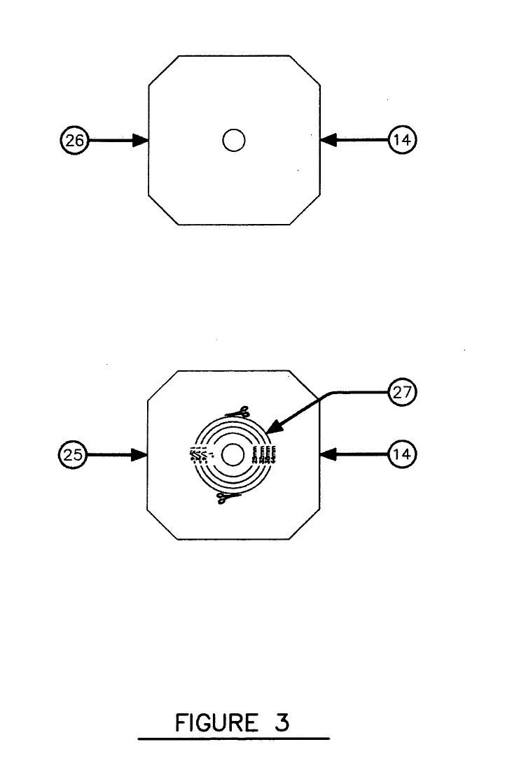

FIG. 4 is the front and back of the insert (disposable recyclable body waste collecting bag or liner).

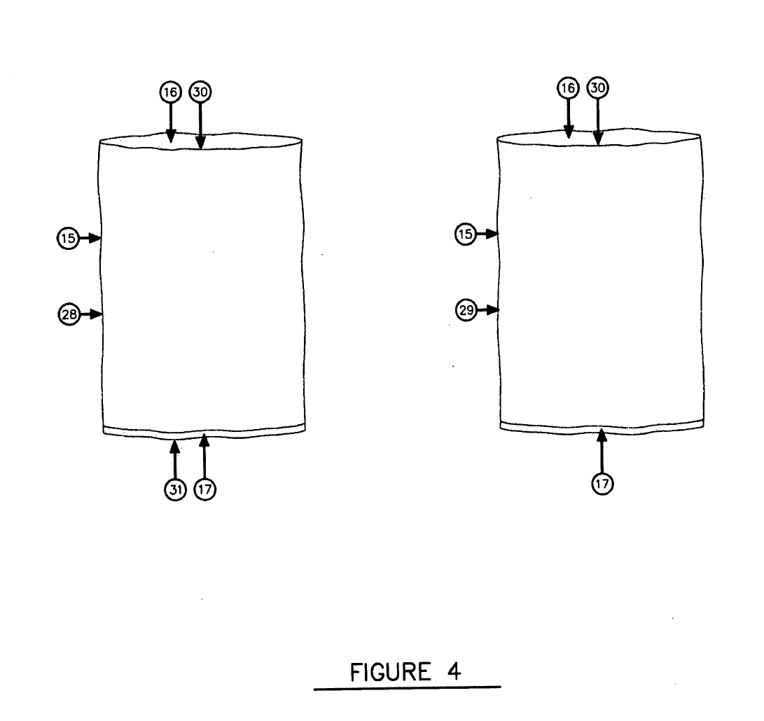

FIG. 5 is the front and back of the snap-on type ring mechanically connecting stoma membrane (diaphragm barrier membrane).

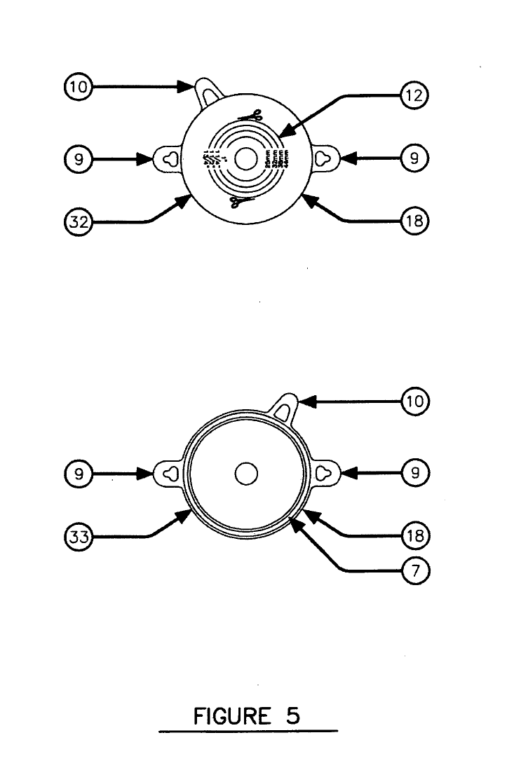

FIG. 6 is the assembly of a two-piece snap-on type ring mechanically connecting colostomy pouch and adhesive wafer or flange, with the stoma diaphragm barrier membrane in place between the colostomy pouch and the adhesive wafer or flange.

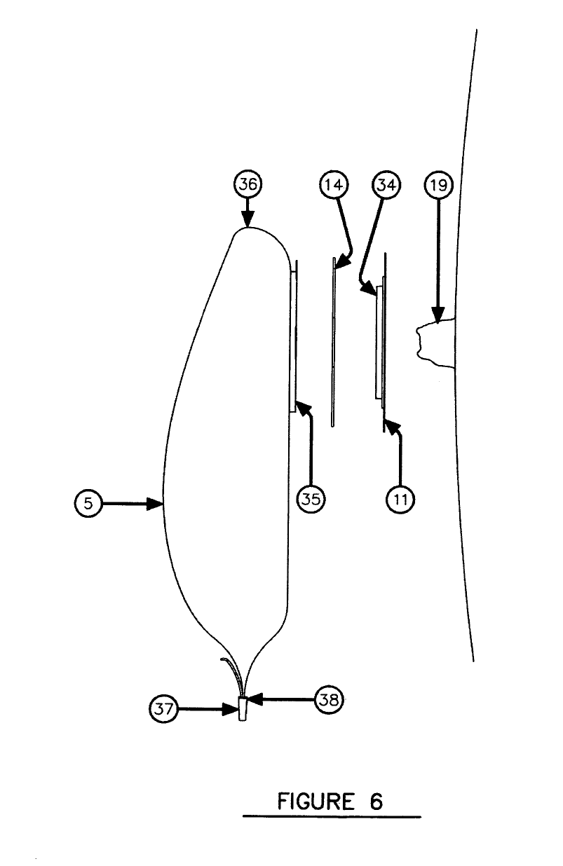

FIG. 7 is the assembly of a two-piece snap-on type ring mechanically connecting colostomy pouch and adhesive wafer or flange, with the stoma diaphragm barrier membrane and the insert (disposable recyclable body waste collecting liner) in place.

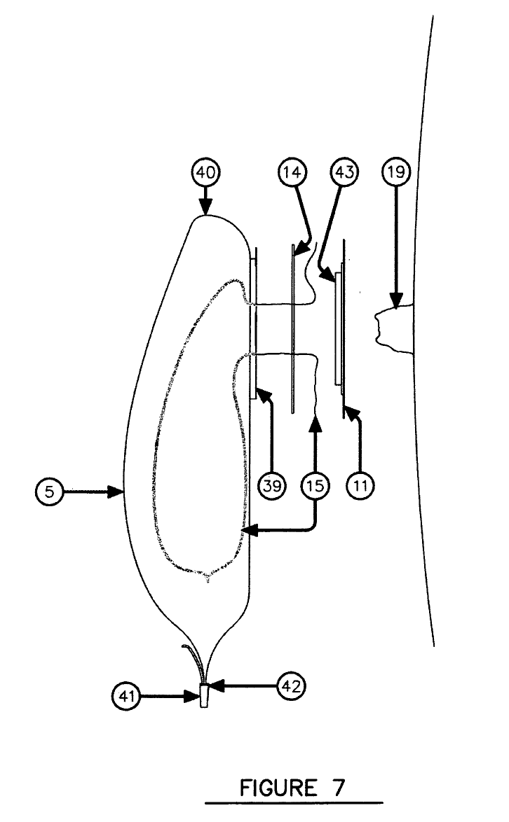

FIG. 8 is the assembly of a two-piece snap-on type ring mechanically connecting colostomy adhesive wafer or flange with the snap-on type ring mechanically connecting stoma diaphragm barrier and the insert (disposable recyclable body waste collecting liner in place).

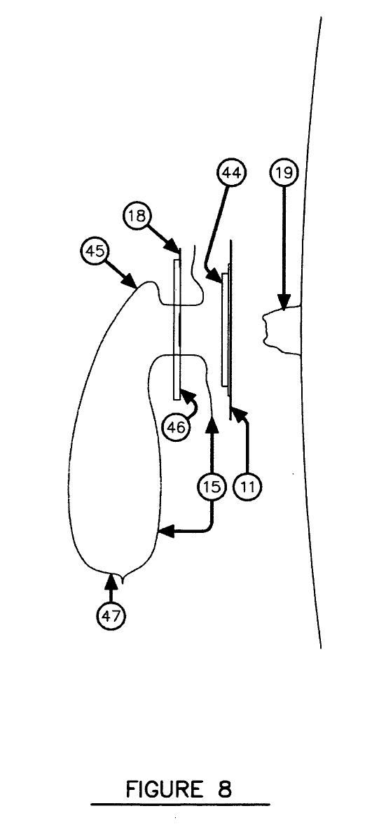

### DRAWINGS DETAILED EMBODIMENT

1) Front
2) Back
3) Top
4) Bottom
5) Colostomy pouch of a two-piece Velcro pouch and separate stoma wafer assembly
6) Velcro
7) Snap-on ring (Female mechanical connection)
8) Snap-on ring (Male mechanical connection)
9) Belt strap handles
10) Holding or handling handle
11) Stoma adhesive wafer
12) Cut-out to fit around a stoma
13) Adhesive
14) Membrane (Stoma diaphragm barrier membrane)
15) Insert (Disposable recyclable body waste collecting bag or liner)
16) Open
17) Shut
18) Membrane (Stoma diaphragm barrier membrane snap-on style)
19) Stoma
20) Velcro
21) Velcro
22) Front
23) Back
24) Adhesive
25) Front
26) Back
27) Cut-out to fit around a stoma
28) Front
29) Back
30) Top
31) Bottom
32) Front
33) Back
34) Back
35) Back
36) Top
37) Bottom
38) Velcro
39) Back
40) Top
41) Bottom
42) Velcro
43) Back
44) Back
45) Top
46) Back
47) Bottom

### DETAILED DESCRIPTION OF THE INVENTION

The present invention is the method and the equipment invented to prevent the contact of body wastes with the skin of the stoma of a colostomy, ileostomy, or urostomy patient by using a stoma membrane (diaphragm barrier) in three manners.

The stoma diaphragm barrier is a thin flat membrane or thin sheet of elastic or other material with an orifice or a hole at the center of it that shall be the same diameter as the diameter of the stoma of a patient and the membrane of the stoma diaphragm barrier surrounding the stoma size orifice or hole at the center of the stoma diaphragm barrier shall be sufficiently larger in circumference than the lip of the snap-on ring of the wafer adhering around the stoma of a patient in order to allow for the folding of the stoma diaphragm barrier membrane over the lip of the stoma wafer and to still have an extra inch (1″) or two inches (2″) of the stoma diaphragm barrier membrane to rest on the skin of a patient or, the prefabricated stoma diaphragm barrier designed by this invention would serve to replace the conventional waste collecting pouch, which is currently attached to the snap-on ring of the body waste collecting pouch assembly, with this invention's stoma diaphragm barrier.

The first part of three parts of the present invention is to describe the equipment and the method of inserting the membrane (stoma diaphragm barrier membrane) between a stoma wafer and a detachable body waste collecting pouch which membrane (stoma diaphragm barrier) is fastened, fitted around and overlapped over the outer circumference of the circular plastic or similar material mechanically connecting snap-on ring of the detachable body waste collecting pouch. The membrane (stoma diaphragm barrier) that is now fastened to the body waste collecting pouch is slid over the stoma of a patient and the ring of the detachable body waste collecting pouch with the overlapping membrane is snapped onto the mechanically connecting ring of the stoma wafer. The membrane (stoma diaphragm barrier membrane) of this invention could be prefabricated to fit onto the snap-on mechanically connecting ring of the body waste collecting pouch which in turn will snap onto the opposite corresponding mechanically connecting ring of the stoma wafer adhering around the stoma of a patient and must be prefabricated with the membrane (stoma diaphragm barrier membrane) in place for use in one piece stoma wafer and pouch assemblies as the membrane (stoma diaphragm membrane) has to be installed permanently between the stoma pouch and the stoma wafer adhering to the skin around the stoma of a patient.

The second part of three parts of the present invention is to describe the equipment and the method of using an insert (toilet disposable recyclable stoma body waste collecting pouch or bag liner) such as the toilet disposable recyclable stoma body waste collecting bag liner described in the U.S. Pat. No. 5,423,782 that is inserted with open end first through the center opening of the membrane (stoma barrier membrane), is pulled through the center membrane stoma barrier membrane) stoma orifice sufficiently to surround and to overlap over the exterior of the rim of the ring of the membrane (stoma diaphragm barrier), the insert (disposable recyclable liner) is then inserted with the closed end first into the standard body waste collecting pouch with attached membrane (stoma diaphragm barrier) in position and is mechanically connected by snapping together between the membrane (stoma diaphragm barrier membrane), the stoma body excrement collecting pouch and the stoma wafer adhering around the stoma of a patient. This sealing method prevents the body wastes from touching the skin. When the insert (toilet disposable recyclable stoma body waste collecting liner) is filled with body waste, the insert (body waste collecting pouch) is detached from the stoma wafer, is removed from the membrane (stoma diaphragm barrier), is flushed in the toilet, is replaced by a fresh insert (toilet disposable recyclable liner) and is re-attached to the wafer adhering around the stoma of a patient

The third part of three parts of the present invention eliminates the use of conventional body waste collecting pouches or bags which cannot be disposed of in a toilet, thus eliminating with time the use of the first and the second part of the three parts of the present invention. The actual stoma body waste collecting pouch or bag of the snap-on ring of the body waste collecting pouch or bag connecting system, is cut out and refitted with the membrane (stoma diaphragm barrier), as it will have been prefabricated in such a way as to allow the patient to pass the insert (disposable recyclable body waste collecting bag or liner) with open end first through the central opening of the membrane (stoma diaphragm barrier), whose diameter has been cut to fit snuggly around the patient's stoma. The insert (recyclable body waste collecting liner) will be pulled sufficiently through the center stoma orifice of the membrane (stoma diaphragm barrier) so as to overlap the insert (disposable recyclable liner) over the outer lip of the snap-on mechanically connecting ring of the membrane (stoma diaphragm barrier) and to snap onto the opposite corresponding stoma wafer that is adhering around the stoma of a patient.

Body wastes come in contact only with the extremity of the stoma that is exposed inside the insert (recyclable disposable liner), as the stoma protrudes partly out of the insert (disposable recyclable body waste collecting pouch liner) which is held snuggly in position by the membrane (stoma diaphragm barrier). The body wastes and gases are prevented from leaking out or from escaping under the membrane (stoma diaphragm barrier). This sealing method achieves the desired result of having the least surface area possible for body wastes to come in contact with the skin as only the front portion of the stoma inside the insert (disposable recyclable liner) touches the body wastes, while the lower portion of the stoma closest to the skin and the skin around it are protected from body wastes, especially body wastes that are in the liquid or semi-solid form. Body wastes are prevented from escaping underneath the membrane (stoma diaphragm barrier) and under the insert (disposable recyclable bag or liner). The front portion of all stomas is not affected by the contact of body wastes as it is resistant to acids and other similar solutions that would normally burn the skin.

For general purposes, the insert (body waste collecting disposable recyclable bag or liner) could be replaced by an impermeable bag that is not discarded that has an opening such as the conventional existing Velcro system that opens and shuts to allow for the emptying of the body wastes that collect, or by a bag with a tube that leads to another bag or another receptacle or collector of body wastes if in the liquid form.

For general purposes, the snap-on method developed by this invention for the purpose of connecting or joining together the stoma body waste collecting pouch or the membrane (stoma diaphragm barrier) which has been prefabricated so that it is a part of the snap-on ring that is snapped on to the stoma wafer adhering around the stoma of a patient, could also be in the form of a clamp-on method, or bottle screw cap method or whatever other method of joining together the stoma body waste collecting pouch or bag to the stoma wafer adhering around the stoma of a patient.

For general purposes the membrane (stoma diaphragm barrier membrane) could be convex or concave shaped depending on the application.

For general purposes the membrane (stoma diaphragm barrier) could be made of flexible material, of rigid material, of foam material, or of a combination of the materials specified.

1. An ostomy appliance for reducing the area of intestinal effluent on a stoma wafer by 90% to 100% comprising: a membrane on a mechanically connecting ring that connects to a corresponding mechanically connecting ring of a wafer such as the mechanically connecting ring of an ostomy pouch and wafer assembly of US patent Bedard et al. US 2003/0023210 A1, wherein said membrane has an orifice at the center, wherein an insert is configured for positioning within said orifice

2. The ostomy appliance of claim 1 wherein said orifice is equal to or smaller in diameter than a stoma.

3. The ostomy appliance of claim 1 wherein said membrane on a mechanically connecting ring is created by replacing the pouch from a ring of a pouch and adhesive wafer assembly that uses a ring to connect together mechanically said pouch to said adhesive wafer such as the pouch and wafer mechanically connecting ring attachment system of US patent Bedard et al. US 2003/0023210 A1, and by permanently installing said membrane flat on said ring from which said pouch has been removed.

4. The ostomy appliance of claim 1 wherein said membrane may vary in thickness from less than about a ten thousandth of an inch to several inches.

5. The ostomy appliance of claim 1 wherein said insert defines an inner chamber wherein said insert is configured to collect said intestinal effluent fluid that flows from said stoma, wherein said insert has:
the open end larger in diameter than the diameter of said ring of said membrane
the closed end equal to or greater than the open end of said insert
the two sides equal to or longer than the diameter of said open end.

6. The ostomy appliance of claim 1 wherein said insert is pliable.

7. The ostomy appliance of claim 1 wherein said insert is impermeable.

8. The ostomy appliance of claim 1 wherein the opposite corresponding adhesive wafer of said mechanically connecting ring ostomy pouch and wafer assembly is installed by adhering to the skin around said stoma.

9. The ostomy appliance of claim 1 wherein said insert is installed by
passing open end first through the top end of said orifice,
proceeding by pulling said insert through the bottom end of said orifice,
and completing by overlapping said insert by ¼ to ½ inches over the top of the outer circumference of said mechanically connecting ring of said membrane.

10. The ostomy appliance of claim 1 wherein said membrane with said insert in overlapped position is slid down said stoma via said orifice.

11. The ostomy appliance of claim 1 wherein said membrane with said insert in overlapped position is slid down said stoma via said orifice and is mechanically connected to the opposite corresponding mechanically connecting ring of said adhesive wafer.
